(19) United States
(12) Patent Application Publication
(10) Pub. No.: US 2017/0217000 A1
(43) Pub. Date: Aug. 3, 2017

(54) COMBINATION SCREWDRIVER AND TORQUE LIMITING SYSTEM

(71) Applicants: Mercator Innovations BVBA, Schilde (BE); Innortho BVBA, Hasselt (BE)

(72) Inventors: Carl Dierickx, Hasselt (BE); Yves Fortems, Schilde (BE); Dirk Wenmakers, Dilsen (BE)

(21) Appl. No.: 15/486,355

(22) Filed: Apr. 13, 2017

Related U.S. Application Data
(63) Continuation-in-part of application No. PCT/EP2015/073655, filed on Oct. 13, 2015.

(30) Foreign Application Priority Data
Oct. 13, 2014 (NL) 2013623

(51) Int. Cl. B25B 23/14; A61B 17/88; A61B 17/16; B25B 21/00; A61B 90/00

(52) U.S. Cl. CPC B25B 23/1415; B25B 21/007; A61B 90/03; A61B 17/1628; A61B 17/8875; A61B 2090/031

(57) ABSTRACT

The present invention relates to the field of combination rotary tools, more in particular in the combination of screwdrivers and drills. In particular, the invention relates to a sleeve member having a hollow portion adapted to embrace a drill, and comprising a bolt- or screw-fitting unit. More in particular, in the context of the present invention said sleeve member is provided with a built-in mechanism for torque limitation.

### CROSS-REFERENCE TO RELATED APPLICATIONS

This application is filed under 35 U.S.C. §111 as a continuation-in-part of International Patent Application No. PCT/EP2015/073655, filed on Oct. 13, 2015, which designates the United States and claims priority to Netherlands Application No. 2013623, filed on Oct. 13, 2014, the contents of which are hereby incorporated by reference in their entirety.

### FIELD OF THE INVENTION

The present invention relates to the field of combination rotary tools, more in particular in the combination of screwdrivers and drills. In particular, the invention relates to a sleeve member having a hollow portion adapted to embrace a drill, and comprising a bolt- or screw-fitting unit. More in particular, in the context of the present invention said sleeve member is provided with a built-in mechanism for torque limitation.

### BACKGROUND TO THE INVENTION

In certain types of surgery and construction works, it is necessary for the operator (surgeon or workman) to drill several holes and subsequently drive fastening screws or bolts into said holes. With conventional devices, the operator needs 2 separate devices, a drill and a separate screwdriver; or when using a single device, the operator, after drilling a hole, needs to change the drill ending on the motor unit with a screwdriver ending before he can fasten a screw or bolt. As evident, this changing of the ends takes time, and can distort the surgical or work flow.

As a solution for this problem, the present invention provides a hollow sleeve with a screw-or bolt-fitting socket/unit on one ending, which is adapted to embrace a drill. This type of slide-on screwdrivers is known for example from DE202010011308, DE2822372, U.S. Pat. No. 4,218,795, U.S. Pat. No. 5,313,680, U.S. Pat. No. 3,932,904, U.S. Pat. No. 3,484,114, EP2345496, U.S. Pat. No. 5,409,333 and U.S. Pat. No. 4,796,319. However, none of these addresses the aspects of torque limitation, let it be to provide a solution to the problems associated therewith.

In particular during surgery and often also in construction works, it is highly important to use a torque limiter to avoid damage to the device or damage to the areas of application of the screws or bolts and possible subsequent ‘cold welding’. Evidently, each type/size of screw or bolt requires a suitably adapted torque limiter. Hence, on top of the changing process from drill to screwdriver, either a pre-installed torque limiter needs to be adjusted or an adjusted and regularly calibrated torque limiter needs to be installed in accordance with the type/size of screw or bolt. This process again takes precious time. Furthermore, when erroneously installing the wrong or non-calibrated torque limiter or erroneously adjusting the pre-installed torque limiter, in accordance with a certain screw or bolt, crucial errors will occur, especially during stressful situations such as urgent surgical procedures.

The present invention provides a solution to these problems by providing a hollow sleeve member with a screw-or bolt-fitting socket/unit on one ending, which is adapted to embrace a drill, and which is provided with a built-in mechanism for torque limitation. In a particular embodiment, said mechanism for torque limitation is adapted, depending on the type of bolt-or screw-fitting unit in said sleeve member. Hence, after drilling a hole, the operator can easily slide the screwdriver part (hollow sleeve) over the drill, and thus quickly switch from drilling to screwdriving, with the correct torque limiter readily available. Although, numerous torque limiting devices are known in the art, these are either integrated in the motor unit (US20110184425, WO2013178932) or provided as a separate unit to be installed (e.g. U.S. Pat. No. 3,167,936, U.S. Pat. No. 3,942,337, U.S. Pat. No. 6,132,435, EP1996371, EP2311397), however, these are not integrated in the screwdriver part, let it be pre-adjusted depending on the bolt- or screw-fitting unit at the end of the screwdriver part.

### SUMMARY OF THE INVENTION

In a first aspect, the present invention provides a sleeve member (1) comprising:
a hollow portion (2) adapted to embrace a drill (13),
a bolt- or screw-fitting unit (3) on a first end (1a) of said sleeve member (1), and
attachment means (4) on a second end (1b) of said sleeve member (1), for releasably
engaging said sleeve member (1) to said drill (13) or to a motor unit (14);
   said sleeve member (1) being characterized in that it comprises a built-in mechanism for torque limitation (5).

In a particular embodiment, said built-in mechanism for torque limitation (5) is adapted, depending on the type of bolt- or screw-fitting unit (3) in said sleeve member (1).

In another particular embodiment, said built-in mechanism for torque limitation (5) is defined by one or more breakpoints (5a) in said sleeve member (1). Hence, said sleeve member is preferably for single use. In said particular embodiment, the resistance of said breakpoints (5a) is preferably adapted depending on the type of bolt- or screw-fitting unit (3) in said sleeve member (1). In this particular embodiment, the sleeve member (1) may be made from any suitable material, however, preferably from metal or plastic, most preferably from a plastic material, such as for example polyethylene (PE), polypropylene (PP), polystyrene (PS), polyether ether ketone (PEEK), . . .

In yet another particular embodiment, said built-in mechanism for torque limitation (5) is defined by one or more detents (5b), preferably at the second end (1b) of said sleeve member (1), said detents (5b) being adapted to receive one or more spring-rolling elements (8) from a torque limiter (11 or 19) mounted on said drill (13) or motor unit (14). In said particular embodiment, the size and/or form of said detents (5b) in said sleeve member (1) is preferably adapted, depending on the type of bolt- or screw-fitting unit (3). Furthermore, said detents (5b) may be asymmetric in shape thereby allowing to apply a different torque limitation in clockwise or counterclockwise direction. In this particular embodiment, the sleeve member (1) may be made from any suitable material such as for example a plastic material, such as polyethylene (PE), polypropylene (PP), polystyrene (PS), polyether ether ketone (PEEK) and the like; or from metal such as for example stainless steel.

In a further aspect the present invention provides a kit comprising a sleeve member (1) according to the present invention, and one or more bolts or screws adapted to fit the bolt-or screw fitting unit (3) on said first end (1a) of said sleeve member. These screws can preferably also be prefitted on the bolt-or screw fitting unit (3).

In yet a further aspect, the present invention provides a torque limiter (11) according to a first embodiment, comprising:
a first torque limiter element (6) capable of embracing at least a first part of a driven member (12)
a second torque limiter element (7) releasably engageable with said first torque limiter element (6)
one or more spring-rolling elements (8) for releasably engaging said second torque limiter element to said driven member (12) by means of a built-in mechanism for torque limitation (5b) present on said driven member (12) or on a sleeve member (1) according to this invention, and capable of embracing at least a second part of said driven member (12).

The torque limiter according to this invention, is capable of embracing any suitable type of driven member (12), such as a screwdriver, drill, . . . In the context of the present invention, the driven member is preferably a drill (13). Hence, and in correspondence with the figures herein after, in the remainder of the description, the specific use of a drill (13) is included, with the understanding that it can be interchanged with any suitable type of driven member (12).

Therefore, in a further aspect, the present invention provides a torque limiter (11) according to the first embodiment, comprising:
a first torque limiter element (6) capable of embracing at least a first part of a drill (13)
a second torque limiter element (7) releasably engageable with said first torque limiter element (6)
   one or more spring-rolling elements (8) for releasably engaging said second torque limiter element to said drill (13) by means of a built-in mechanism for torque limitation (5b) present on said drill (13) or on a sleeve member (1) according to this invention, and capable of embracing at least a second part of said drill (13).

In yet a further aspect, the present invention provides a torque limiter (19), according to a second embodiment, comprising:
a first torque limiter element (6) capable of embracing at least a first part of a drill (13);
a second torque limiter element (7) for releasably engaging said first torque limiter element (6) with said drill (13);
one or more spring-rolling elements (8) for releasably engaging said drill (13) with said second torque limiter element by means of a built-in mechanism for torque limitation (5b) present on said drill (13) or on a sleeve member (1) according to any one of claim 1, 2, 5, 6, 7, 8 or 10, capable of embracing at least a second part of said drill (13);
a coupling system (16) including a coupling bit (16a) for coupling said torque limiter to a motor unit; and
a locking system (16b) for releasably engaging said drill (13) to said torque limiter (19), by means of a release system (18).

In a particular embodiment, the invention provides a torque limiter as defined herein above, wherein said second torque limiter element (7) comprises a groove to receive a first part (8a) of said one or more spring-rolling elements (8).

In a further embodiment, the present invention provides a combination of a torque limiter according to this invention, with a sleeve member (1) comprising a hollow portion (2) to embrace a drill (13), and releasably engages said second torque limiter element (7) to said drill (13) by means of said one or more spring-rolling elements (8) with the built-in mechanism for torque limitation (5) present on said sleeve member (1) in an indirect way. In said particular embodiment, the built-in mechanism for torque limitation (5) present on said sleeve member (1) preferably comprises one or more detents (5b) adapted to receive a second part of said spring-rolling elements (8b).

In yet a further embodiment, said one or more spring-rolling elements (8) each comprise 2 transmission balls (9) connected by an elastic element (10) such as for example one or more spring washer (10a) or a coil spring.

In yet a further aspect, the present invention provides a sleeve member (1) comprising:
A hollow portion (2) adapted to embrace a drill (3); said hollow portion comprising:
   An external tube (21) having a cam (22)
   An axially moveable sleeve (23) comprising lock pins (24) and release pins (25);
   An elastically deforming body (26) comprising a spline shaft (27) at a first end thereof, which fits within said axially moveable sleeve (23); and a first hollow tube (28) within a smaller second tube (29) at the second end thereof, which fits within said external tube (21); said body (26) is provided with weaknesses (30) which allow adjusting the torsion stiffness of the body (26);
A bolt-or-screw-fitting unit (3) on a first end (1a) of said sleeve member (1); said unit comprising lock connections (31) which may engage with the lock (24) or release pins (25) of the axially moveable sleeve (23)
Attachment means (4) on a second end (1b) of said sleeve member (1) for releasably engaging said sleeve member (1) to a drill (13)
   wherein said sleeve member (1) is characterized in that it comprises a built-in mechanism for torque limitation (5).

In yet a further embodiment, said driven element (12) may be a drill (13) or a screwdriver, preferably a drill, more in particular a drill which may be embraced by a hollow sleeve (the sleeve member (1)), which serves the purpose of a screwdriving element.

In yet a further embodiment, all components of the torque limiter (11) according to this invention can be sterilized by autoclaving without affecting the operation of the device.

The torque limiter (11) according to this invention is in particular suitable for surgical use.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1A: Represents an embodiment of an uninstalled sleeve member (1) according to the invention, wherein the built-in mechanism for torque limitation (5) is defined by one or more breakpoints (5a) in said sleeve member. FIG. 1A shows a possible representation of a collar (15) which grips around said sleeve member (1) as well as a possible representation wherein the hollow portion (2), and bolt-or screw-fitting socket (3) are made from different types of material, e.g. plastic and metal (stainless steel) respectively.

FIG. 1B: Represents an embodiment of an uninstalled sleeve member (1) according to the invention, wherein the built-in mechanism for torque limitation (5) is defined by one or more detents (5b) in said sleeve member.

FIG. 2A: Represents an exemplary type of drill (13) suitable to be used in connection with the sleeve members (1) of the present invention, wherein the drill is further provided with a type of coupling (13b) for easy attachment to a torque limiter (11 or 19) according to this invention.

FIG. 2B: Represents an exemplary type of drill (13) suitable to be used in connection with the sleeve members (1) of the present invention.

FIG. 2C: Represents an exemplary sleeve member (1) according to this invention, when installed on a suitable drill (13) and further (optionally) comprising a collar (15) around said sleeve member.

FIG. 2D: Represents an exemplary sleeve member (1) according to this invention, when installed on a suitable drill (13) wherein the hollow portion (2), and bolt-or screw-fitting socket (3) are made from different types of material, e.g. plastic and metal (stainless steel) respectively.

FIG. 3: Represents, two exemplary types of breakpoints (5a) suitable to be used in the sleeve member according to the present invention, and suitable to be used as the mechanism for torque limitation (5).

FIG. 4: Represents a specific embodiment of the sleeve member (1) of the present invention, wherein the detents (5b) in the sleeve member (1) are adapted on the type/size of the bolt-or screw-fitting unit on the first end (1a) of said sleeve member. The detents can be asymmetrical to provide a different torque limit in both the clockwise and counter-clockwise direction.

FIG. 5: Represents a suitable attachment means (13b) for releasable engaging the drill (13) to a torque limiter (11) according to this invention; and indicates the process of coupling the individual elements: drill (13), torque limiter (11) and hollow sleeve member (1).

FIG. 6A: Represents a first embodiment of a torque limiter (11) according to the present invention, more in particular used in the presence of a coupling system (attachment means 13b) present on the drill (13) or driven member (12), i.e. directly coupled to the drill or driven member.

FIG. 6B: Represents a second embodiment of a torque limiter (19) according to the present invention, more in particular wherein the torque limiter itself comprises a release system for coupling to the drill (13) or driven member (12).

FIG. 7A: Represents the assembly of: a torque limiter (11) with a drill (13) and hollow sleeve member (1) according to this invention, wherein the drill is provided with attachment means (13b) for releasably engaging the drill to the torque limiter, and wherein the torque limiter is provided with a release system (18) for releasably engaging the drill to the torque limiter.

FIG. 7B: Represents the assembly of: a torque limiter (11) with a drill (13) and hollow sleeve member (1) according to this invention, and wherein the torque limiter is provided with a release system (18) for releasably engaging the drill to the torque limiter.

FIG. 8A: Represents an embodiment of the elastic element (10) of the torque limiter, in particular of the double-transmission (8a, 8b) ball system, including spring washers (10a) and transmission balls (9).

FIG. 8B: Represents details of the association of the spring-rolling elements (8) of the torque limiter (11 or 19) of this invention with the detents (5b) in the second end (1b) of the sleeve member according to this invention.

FIG. 9: Represents an embodiment of a kit comprising a sleeve member (1) and a screw or bolt, suitable to engage with the bolt-or screw fitting unit (3) at the first end (1a) of said sleeve member (1), optionally further comprising a barcode (17) or other means for easy identification of the torque limit and mounted screw or bolt.

FIG. 10: Represents a third embodiment of a sleeve member according to the present invention; comprising an external tube (21) with a cam (22) and an axially moveable sleeve (23). Torque limitation by means of this embodiment is based on elastic deformation of an elastically deforming body (26) as detailed further on.

FIG. 11A: Represents close-ups of different parts of the third embodiment of the present invention. FIG. 11A shows the cooperation of the different parts of the third embodiment.

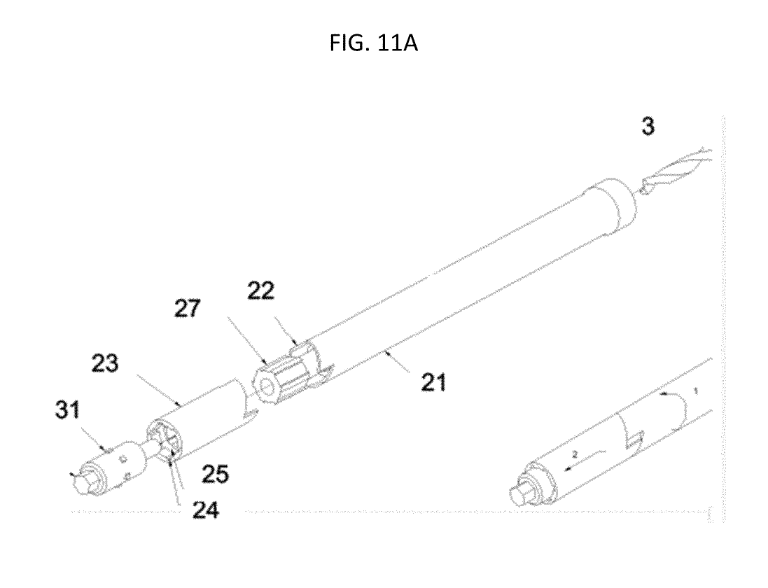

FIG. 11B: Represents close-ups of different parts of the third embodiment of the present invention.

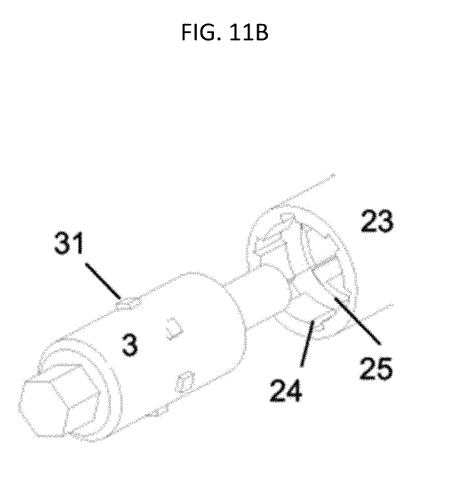

FIG. 11C: Represents close-ups of different parts of the third embodiment of the present invention. FIG. 11C shows the engagement of the axially moveable sleeve (23) with the screw-or-bolt-fitting unit (3); wherein depending on the amount of torque, the lock connections (31) on the screw-or-bolt-fitting unit (3) may either engage with the lock pins (24) on the axially moveable sleeve (23) or may disengage therefrom and align with the release pins (25) of the axially moveable sleeve (23). FIG. 11C shows the engagement of the axially moveable sleeve (23) with the spline shaft (27) on the one hand and with the screw-or-bolt-fitting unit (3) on the other hand.

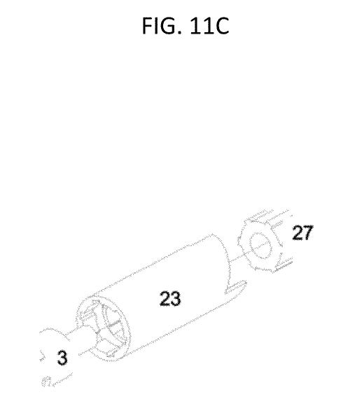

### DETAILED DESCRIPTION OF THE INVENTION

Numbering:

(1) Sleeve member
(1a) First end of sleeve member
(1b) Second end of sleeve member

(2) Hollow portion

(3) Bolt-or screw-fitting unit/socket

(4) Attachment means for releasably engaging sleeve member to drill or motor unit

(5) Built-in mechanism for torque limitation
(5a) Breakpoints in sleeve member
(5b) Detents or recesses in sleeve member

(6) First torque limiting element
(6a) Spring element for displacing second torque limiting element

(7) Second torque limiting element
(7a) Groove in second torque limiting element

(8) Spring-rolling elements
(8a) First part of spring-rolling elements
(8b) Second part of spring-rolling elements

(9) Transmission ball(s)

(10) Elastic element
(10a) Spring Washers

(11) Torque limiter according to the first embodiment

(12) Driven member

(12a) attachment means for releasable engaging the driven member to the motor unit.

(12b) attachment means for releasable engaging the driven member to the torque limiter

(13) Drill

(13a) attachment means for releasable engaging the drill to the motor unit

(13b) attachment means for releasable engaging the drill to the torque limiter.

(14) Motor unit

(15) Collar

(16) Coupling system
(16a) Coupling bit
(16b) Locking System

(17) Identification code (e.g. barcode)

(18) Release System, such as for example spring-rolling elements
(18a) First part of spring-rolling elements
(18b) Second part of spring-rolling elements

(19) Torque limiter according to second embodiment

(20) Torque limiter according to third embodiment

(21) External Tube

(22) Cam

(23) Axially Moveable Sleeve

(24) Lock Pins

(25) Release Pins

(26) Elastically deforming body

(27) Spline Shaft

(28) First Hollow Tube

(29) Second Tube

(30) Weaknesses

(31) Lock Connections

As indicated herein before, the present invention in particular aims at providing a system capable of quickly switching between drilling and screw-driving, while at the same time having a torque limiting system installed, which is adapted to the screw or bolt that is to be used in connection with the drilled hole.

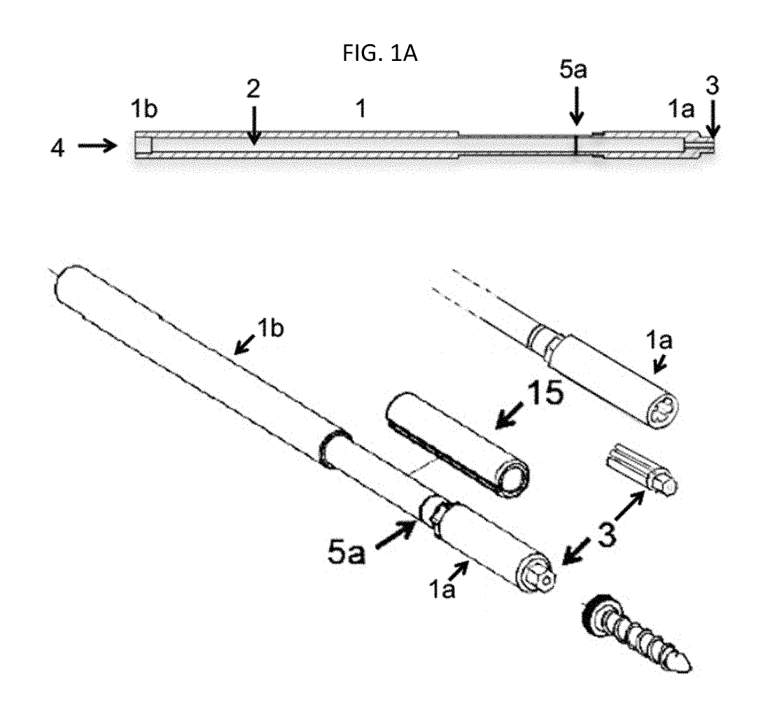

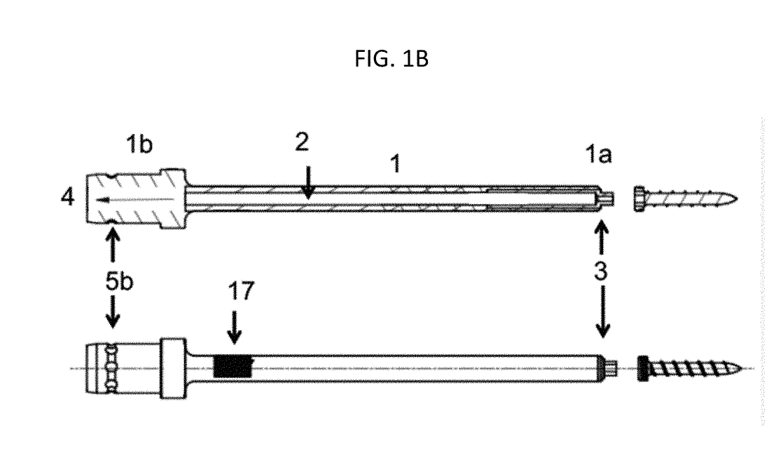

Thereto, in a first aspect, the present invention provides a sleeve member (1) comprising:
a hollow portion (2) adapted to embrace a drill (13),
a bolt- or screw-fitting unit (3) on a first end (1a) of said sleeve member (1), and
attachment means (4) on a second end (1b) of said sleeve member (1), for releasably engaging said sleeve member (1) to said drill (13) or to a motor unit (14);
   said sleeve member (1) being characterized in that it comprises a built-in mechanism for torque limitation (5, in particular 5a and 5b) (see FIGS. 1A and B).

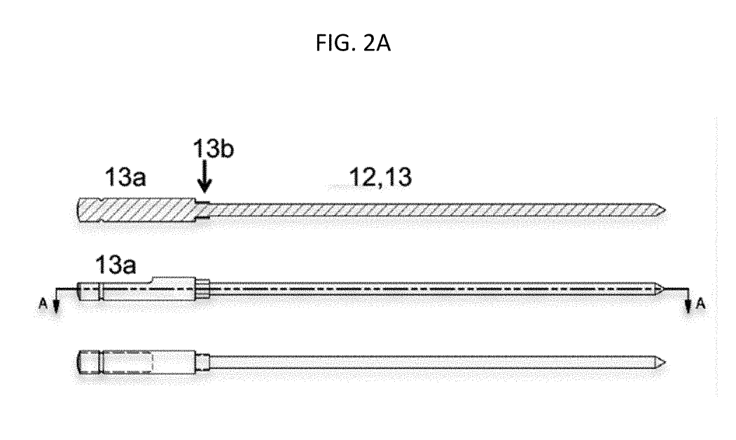

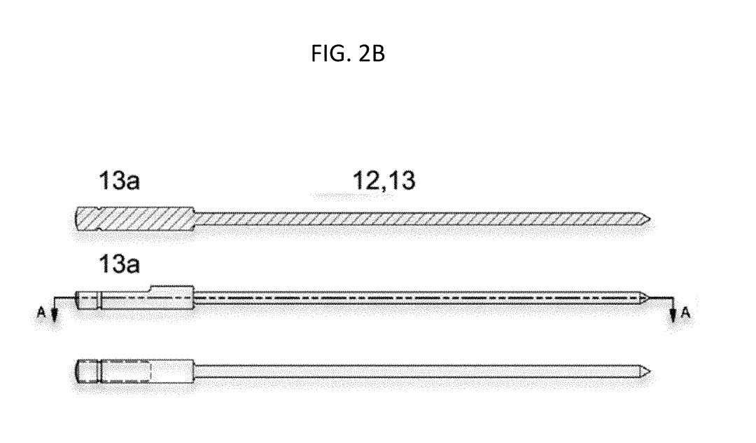

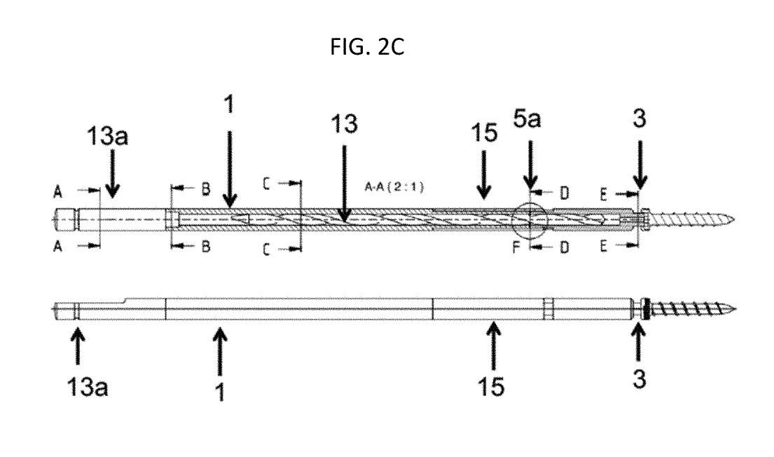

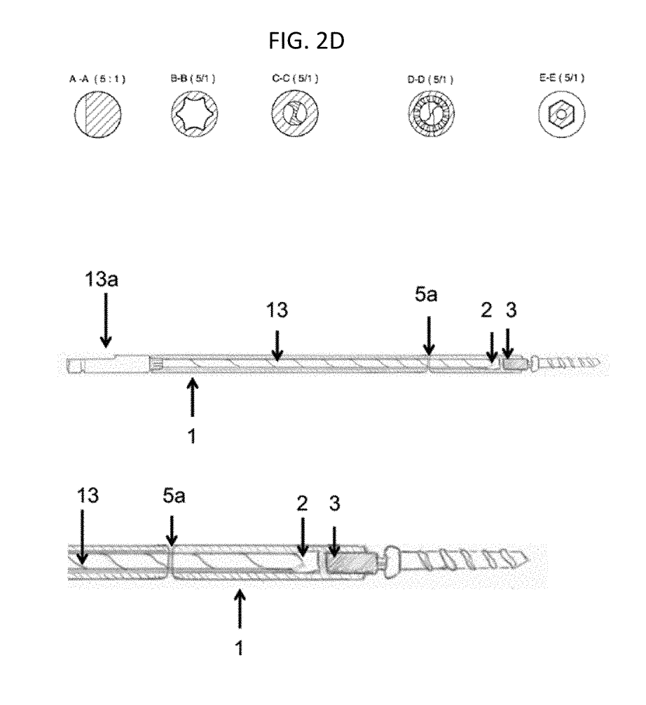

By using this particular type of sleeve member (1), the operator can drill a hole using a motor unit fitted with a drill (13). Subsequently and without removing the drill, the operator can easily slide the hollow sleeve member (1) according to the invention over the drill (as for example illustrated in FIG. 2C), and drive the same motor unit (14) to attach a bolt or screw capable to fit in the bolt-or screw-fitting unit (3) on the first end (1a) of said sleeve member. In addition, the operator should not worry about attaching a suitable torque limiter, or adjusting a pre-installed torque limiter, in accordance with the particular type/size of screw or bolt to be installed, since the sleeve member (1) according to this invention is already provided with a built-in mechanism for torque limitation (5). Hence, as evident, this type of sleeve member (1), significantly reduces the amount of time needed to switch from drilling to screw-driving with the correct torque limiter installed. This gain of time can in particular be crucial during an emergency surgical procedure.

It should be understood that the invention is not limited to motorized assemblies, and that the hollow sleeve member of the present invention, may also be used in association with non-motorized assemblies such as for example, classical screwdriver handles either or not with a quick coupling system, such as currently used in surgery and for example illustrated in WO2005120371.

In the context of the present invention, the terms ‘screw’ or ‘bolt’ are used to define a fastener capable of positioning objects, or holding objects together. For example, these may be used during osteosynthesis, wherein during a surgical procedure a connection must be made between 2 bones or bone fragments using plates, screws, bolts, . . .

The term bolt- or screw-fitting unit (3) as used herein, is defined as an ending on the sleeve member of the present invention, capable of receiving a bolt or screw, and adapted such that the driving action of a motor unit results in driving said bolt or screw into the object.

In the context of the present invention, the term ‘motor unit’ (14) refers to the driving means to which a drill bit and/or screwdriver bit can be fixed, and which is used for the automatic drilling or screwdriving action.

In the context of the present invention, the term ‘drill bit’ refers to the drill ending to be fitted on a motor unit and used to perforate an object such as for example a bone. Similar, a ‘screwdriver bit’ refers to the screwdriver ending to be fitted on the motor unit and used to drive a screw or bolt into a hole.

Evidently, each type/size of screw or bolt requires its own adapted torque limiter, thereby allowing the correct torque to be exerted on said particular type of screw or bolt. Hence, in a particular embodiment, the built-in mechanism for torque limitation (5) is adapted, depending on the type of bolt- or screw-fitting unit (3) in said sleeve member (1). In said embodiment, only screws or bolts that fit in/on the particular bolt- or screw-fitting unit (3) can be used in connection to a particular sleeve member (1), having the corresponding built-in mechanism for torque limitation (5) in place. This embodiment therefore significantly reduces or even removes the error margin of selecting a wrong torque limiter for a particular type of screw or bolt.

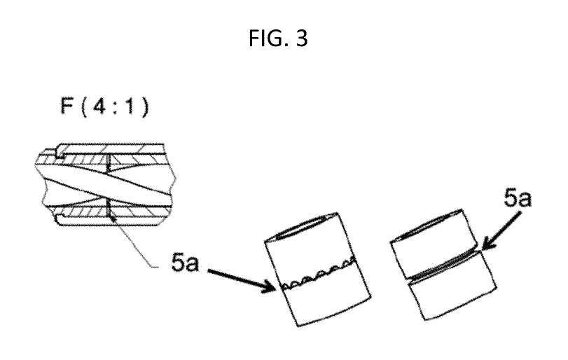

In a particular embodiment, said built-in mechanism for torque limitation (5) is defined by one or more breakpoints (5a) in said sleeve member (see FIGS. 1A and 3). Any suitable means of providing such breakpoints between two or more parts of said sleeve member is possible, such as for example one or more shear pins, one or more weaker areas in the sleeve member, one or more holes in the sleeve member, . . . (see FIG. 3) While operating the sleeve member, the breakpoints will break and disconnect the two or more parts of said sleeve member, at a certain amount of torque asserted on said breakpoint. By using this type of torque limiting mechanism, a new sleeve member (1) comprising intact breakpoints needs to be used for each screw or bolt to be installed, since once the breakpoints (5a) have reached their maximum torque, and were broken, the sleeve member (1) can not be used again. Hence, this embodiment is particularly suitable for single-use applications, wherein a new sleeve member (1) is used for each new screw or bolt to be installed. Therefore, although the sleeve member may be made from any suitable material, such as for example metal (e.g. stainless steel), it is preferably made from a cheap (single-use) material such as a plastic material, for example polyethylene (PE), polypropylene (PP), polystyrene (PS), polyether ether ketone (PEEK) and the like. Alternatively, the sleeve member may be made from two or more different materials. For example, the hollow portion (2) may be made from a plastic material, while the bolt-or screw-fitting socket may be made from metal (e.g. stainless steel). This particular embodiment is further illustrated in FIGS. 1A and 2D. Herein the hollow portion (2), and the built-in mechanism for torque limiting (5a) are made from a plastic material, whereas the bolt-or screw-fitting socket (3) is made from metal (e.g. stainless steel) and fits in the end of the sleeve member (1).

In this particular embodiment, the resistance of said breakpoints (5a) or pins is preferably adapted, depending on the type of bolt- or screw-fitting unit (3) in said sleeve member (1). Hence, as indicated above, only screws or bolts that fit in/on the particular bolt- or screw-fitting unit (3) can be used in connection to a particular sleeve member (1), wherein the resistance of the breakpoints (5a) is adapted based on said particular screws or bolts.

In yet a further embodiment, the sleeve member (1) according to the present invention, may comprise a collar (15), around a part of the sleeve member (1), by which the operator may hold the device in the correct position (see FIG. 1A). In addition, said collar (15) may provide protection for the environment against possible debris coming from breaking of the breakpoints of the built-in mechanism for torque limitation (5a). This is particularly relevant during surgery, wherein it is inacceptable that debris coming from the device would end up in the wound of the patient. When relevant, the collar may also serve the purpose of keeping both parts of the sleeve member together after breaking of the breakpoints. The collar (15) may be rotationally fixed to the sleeve member or may rotate around the sleeve member, in order for the operator to firmly hold the collar, while still allowing rotation of the sleeve member itself.

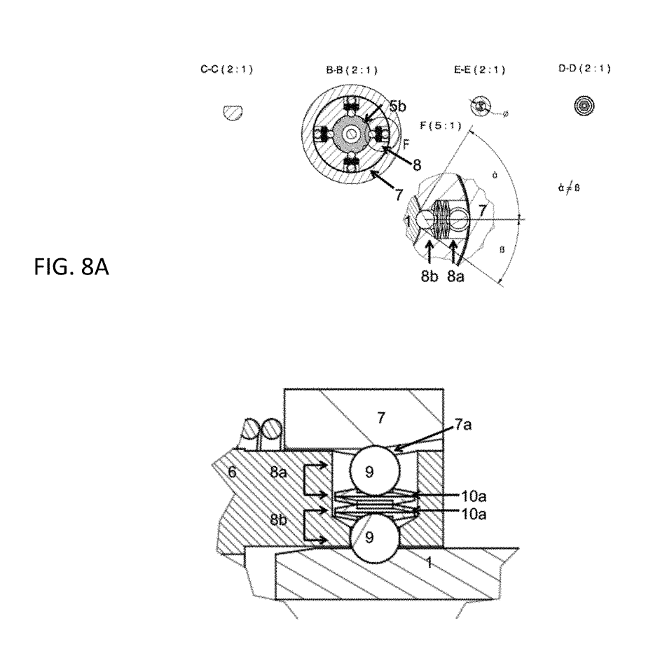

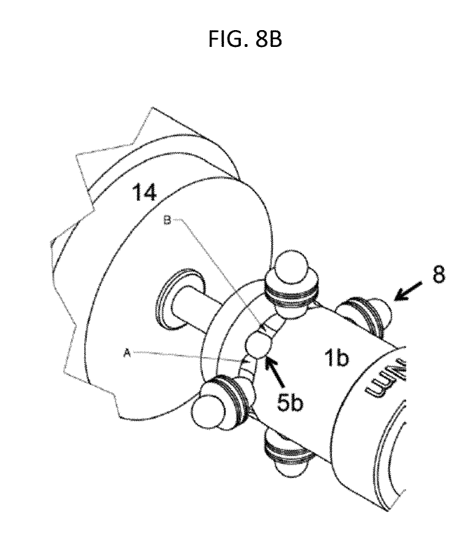

In yet another particular embodiment, the built-in mechanism for torque limitation (5) is defined by one or more detents or recesses (5b) in said sleeve member (1), preferably in the shaft of said sleeve member (see FIG. 1B). These detents (5b) are adapted to receive one or more spring-rolling elements (8) from a torque limiting device mounted on said drill (13) or motor unit (14) (see FIG. 8B). In this particular embodiment, the attachment means (4) are fully integrated with and correspond to the built-in mechanism for torque limitation (5) of the sleeve member (1). In this embodiment the sleeve member (1) may be made from any suitable material such as for example a plastic material, such as polyethylene (PE), polypropylene (PP), polystyrene (PS), polyether ether ketone (PEEK) . . . ; or from metal such as stainless steel. Alternatively, the sleeve member may be made from two or more different materials. For example, the hollow portion (2) may be made from a plastic material, while the bolt-or screw-fitting socket may be made from metal (e.g. stainless steel).

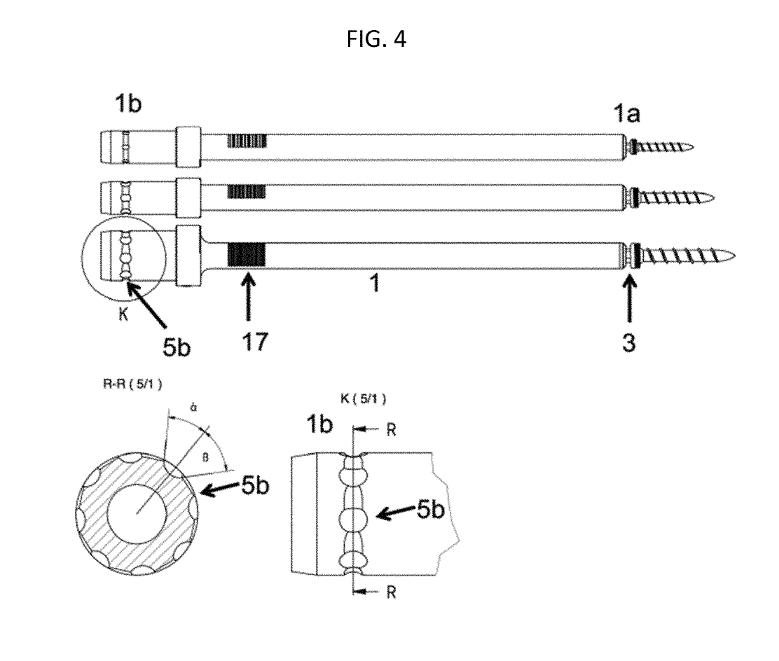

In a particular embodiment, the size and/or form of said detents or recesses (5b) may be adjusted depending on the type of bolt- or screw-fitting unit (3) in said sleeve member (1) and are thus in fact adjusted on the type/size of the bolt or screw to be installed. This is for example illustrated in FIG. 4. In said instance, each type of screw or bolt has its own adapted sleeve member (1) with appropriately adapted detents (5b). Hence, the torque limiting device mounted on a drill (13) or motor unit (14) and comprising spring-rolling elements (8) for engaging with said detents, may be kept standard amongst different types of screws or bolts, whereas the adjustment in relation to the type of screw or bolt is achieved by the adapted detents (5b) in the specific sleeve members (1). This is in contrast to conventional devices, wherein the torque limiting device needs to be adjusted or interchanged for each type of screw or bolts to be installed. Hence, this embodiment allows the use of a single torque limiting device in association with a set of different screws and bolts each necessitating a specifically adjusted torque limiter.

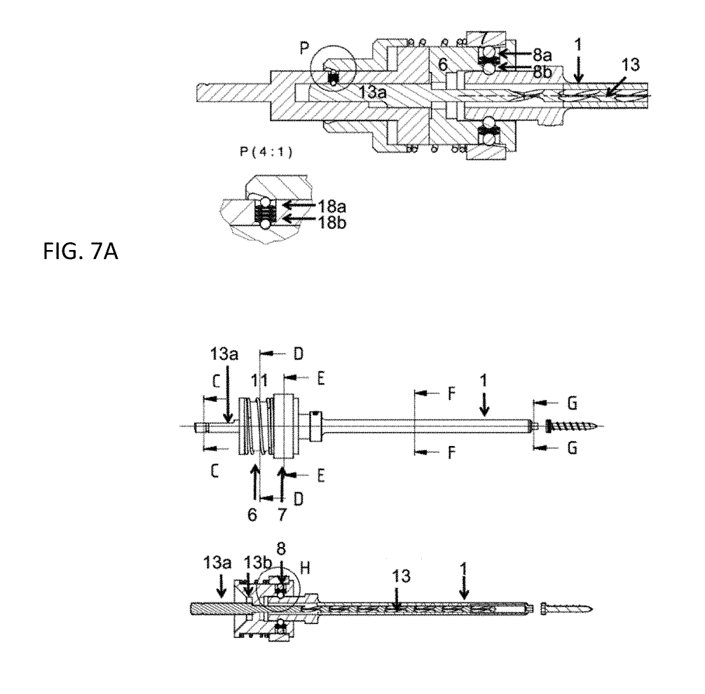

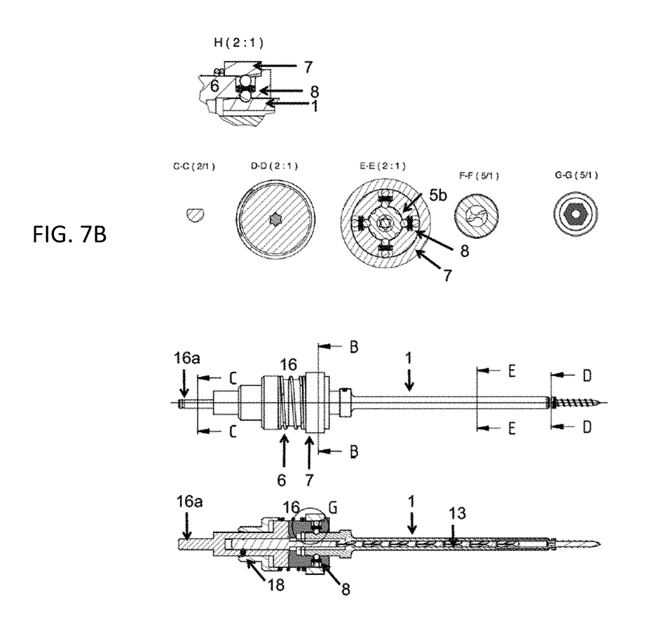

In a particular embodiment, the detents (5b) in said sleeve member (1) may be symmetric in size or shape thereby allowing to apply a similar torque limitation in clockwise or counterclockwise direction. Alternatively the detents (5b) in said sleeve member (1) may be asymmetric in size or shape thereby allowing to apply a different torque limitation in clockwise or counterclockwise direction. Preferably, the detents (5b) are asymmetric in the rotational direction (i.e. bi-directional), thereby allowing a different maximum amount of torque in the clockwise or counterclockwise direction (see FIGS. 4 and 7B, i.e. □≠□). In said instance, the detents (5b) are designed such that the torque to be transmitted is dependent on the direction of rotation. For example, when rotating the hollow sleeve (1) in the clockwise direction, i.e. fastening direction, the maximum amount of torque to be allowed (i.e. torque tolerance) on the device is preferably smaller in comparison with rotating the hollow sleeve in the counter-clockwise direction, i.e. unfastening direction. As such, although the maximum amount of torque may be reached in the clockwise direction, and further tightening the screw or bolt is impossible, it still remains possible to untighten the screw or bolt due to the higher torque tolerance in the counter-clockwise direction.

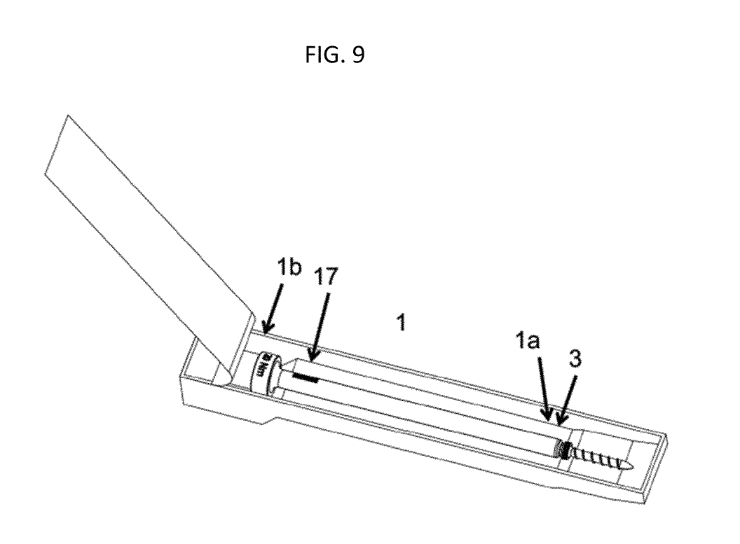

In a further aspect, the present invention provides a kit comprising a sleeve member (1) according to this invention, and one or more screws or bolts adapted to fit said bolt- or screw-fitting unit (3) on said first end (1a) of said sleeve member, such as for example illustrated in FIG. 9. Hence, in said embodiment, the sleeve member and screws/bolts are packed together such that at all instances, the correct screws/bolts are associated with the corresponding bolt- or screw-fitting socket/unit (3) and correct built-in system for torque limitation (5). This embodiment thus provides a particularly suitable combination for use during surgery, and significantly reduces the error-margin associated with the incorrect combination of screws/bolts and possible non-calibrated torque limiting systems.

The advantages of this particular embodiment during a surgical procedure become particularly evident when comparing the original process of changing between drill and screwdriver, with the new process as provided herein:

| Original Process | Process of Invention |
|---|---|
| Place drill on motor unit | Place drill on motor unit |
| Drill hole in bone/plate | Drill hole in bone/plate |
| Measure depth of hole | Measure depth of hole |
| Take screw/bolt out of sterile box | Assembling correct screw-hollow |
| | sleeve member combination, or |
| | receive the same from |
| | assistants/nurses, in sterile condition |
| | Take sleeve member of the invention |
| | with |
| Measure length of screw/bolt | |
| Remove drill bit from motor unit | |
| Attach correct torque limiter on | |
| motor unit | |
| Place screwdriver bit on torque | |
| limiter | |
| Place screw on screwdriver bit | Slide sleeve member of the invention |
| | over drill |
| Introduce screw/bolt in bone | Introduce screw/bolt in bone |
| Remove screwdriver bit from | Slide screwdriver of drill |
| motor unit | |
| Remove torque limiter from motor | |
| unit | |

Hence by using the sleeve member (1) of the present invention, at least 5 steps of the original process can be skipped. Experiments have shown that with the current invention, a significant reduction in operation time during a surgical procedure can be achieved.

The use of such a kit according to the invention also allows traceability during a surgical procedure. For example, when using a unique identification code (17) in association with each package, it is possible to track the actually used screws/bolts during and after the operation, at all instances. Such code may for example be located on the package and/or be applied on the side of the hollow sleeve, such as for example on the collar, whenever present. As an exemplary code, a barcode (17) may be used, which can be scanned during a surgical procedure before or after the application of the associated screw or bolt. Alternatively a removable label, either or not comprising such barcode, may be used. Said removable label may then be removed from the package or hollow sleeve during surgery and included in the patient's medical notes. Furthermore, the package, sleeve member and/or parts thereof, may also be provided with a colour code, for easily identifying the type/size of screw or bolt, which may also be provided with a colour code, to be installed; and/or the torque to be applied (e.g. expressed in Nm).

To allow the quick changing between drilling and screw-driving while simultaneously providing a corresponding torque limiter for each type of screw or bolt, the inventors have further developed a novel type of torque limiter (11 or 19) particularly suitable in connection with the sleeve member (1) as described herein above.

Hence, in a further aspect, the present invention provides a torque limiter (11) comprising:
a first torque limiter element (6) capable of embracing at least a first part of a driven member (12), more specifically as drill (13)
a second torque limiter element (7) releasably engageable with said first torque limiter element (6)
one or more spring-rolling elements (8) for releasably engaging said second torque limiter element to said driven member (12) by means of a built-in mechanism for torque limitation (5) present on a sleeve member (1) according to this invention, and capable of embracing at least a second part of said driven member (12).

As already indicated hereinabove, the torque limiter according to this invention, is capable of embracing any suitable type of driven member (12), such as a screwdriver, drill, . . . In the context of the present invention, the driven member is preferably a drill (13).

Therefore, in a further aspect, the present invention provides a torque limiter (11) according to the first embodiment, comprising:
a first torque limiter element (6) capable of embracing at least a first part of a drill (13)
a second torque limiter element (7) releasably engageable with said first torque limiter element (6)
one or more spring-rolling elements (8) for releasably engaging said second torque limiter element to said drill (13) by means of a built-in mechanism for torque limitation (5b) present on said drill (13) or on a sleeve member (1) according to this invention, and capable of embracing at least a second part of said drill (13).

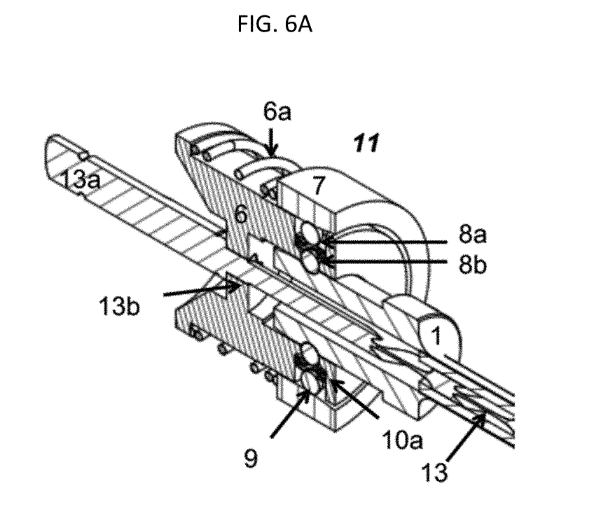

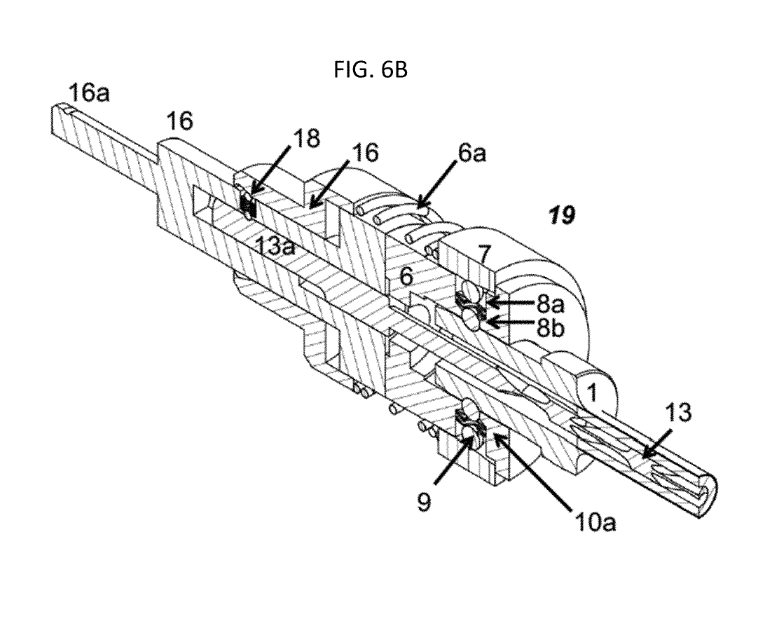

This type of torque limiter is for example illustrated in FIGS. 6A and B.

In a more specific embodiment, the present invention provides as a torque limiter (19) comprising:
a first torque limiter element (6) capable of embracing at least a first part of a drill (13)
a second torque limiter element (7) for releasably engaging said first torque limiter element (6) with said drill (13);
one or more spring-rolling elements (8) for releasably engaging said drill (13) with said second torque limiter element by means of a built-in mechanism for torque limitation (5b) present on said drill (13) or on a sleeve member (1) according to any one of claim 1, 2, 5, 6, 7, 8 or 10, capable of embracing at least a second part of said drill (13).
a coupling system (16) including a coupling bit (16a) for coupling said torque limiter to a motor unit; and a locking system (16b) for releasably engaging said drill (13) to said torque limiter, (19), by means of a release system (18).

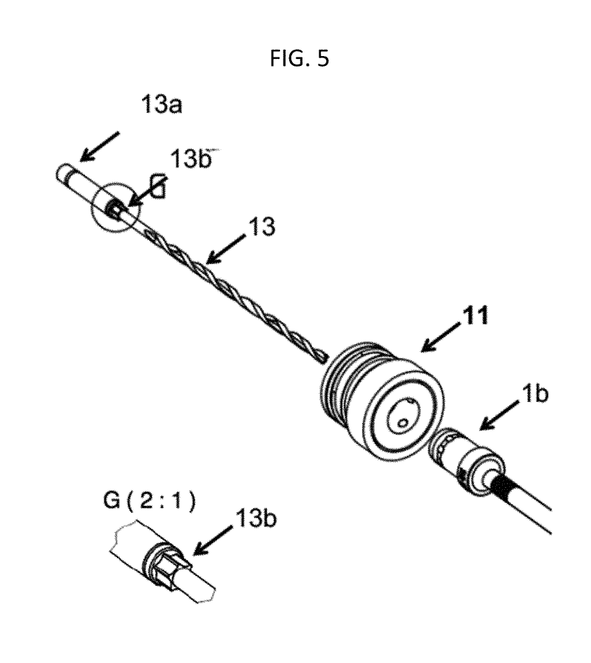

Regardless of the built-in mechanism for torque limitation (5), the connection between the torque limiter (11 or 19) and driven member (12), drill (13) or motor unit (14) may be made by any suitable means, in as long as the torque limiter (11 or 16) is attached with the required rotational fixation to the driven member (12), drill (13) or motor unit (14). For example, the drill (13) may comprise attachment means (13b) having a specific shape and/or form (13a), capable of accommodating the torque limiter (11 or 19), and preventing the first torque limiting element (6) from rotating around its longitudinal axis (see FIGS. 5 and 6A). Such attachment means (13b) may for example be represented by a star-coupling capable of engaging with a corresponding part of the torque limiter. Furthermore, the drill bit (13a) is preferably asymmetric, such that it can only fit in 1 direction in the motor unit (14). In a specific embodiment, said asymmetric ending may be duplicated in the shaft of the drill (13) or driven member (12) to embrace the first torque limiting element (6), and hence serving the purpose of attachment means (13b). As such, also said first torque limiting element (6) may only fit in 1 direction around the drill (13) or driven member (12), and at the same time provides for the required rotational fixation of the torque limiter (11).

Alternatively, the torque limiter may be coupled to the drill (13), or driven member (12) by using a further coupling system (16) as part of the torque limiter (19) as illustrated in FIG. 6B. Hence, in a particular embodiment, the torque limiter (19) according to this invention further comprises a coupling system (16) including a coupling bit (16a) for coupling said torque limiter to a motor unit; and a locking system (16b) for releasably engaging a drill (13) or driven member (12) to said torque limiter (19), by means of a release system (18).

In said embodiment, the torque limiter (19) according to the present invention, may be connected to the driven member (12), in particular a drill (13), by means of a coupling system (16) preferably as part of the torque limiter, as illustrated in FIG. 6B. Such coupling system (16) preferably comprises a coupling bit (16a) for installing it on the motor unit and is adapted to embrace the driven member (12) or drill (13). In this embodiment, the first torque limiting element (6) may be part of, or attached to the coupling system (16). The drill (13) or driven member (12) may be engaged to the coupling system by means of a release system (18), such as for example one or more further spring-rolling elements.

Regardless of the type of torque limiter (11 or 19), the hollow sleeve member of the invention (1) may be installed on the torque limiter (11 or 19) by means of the spring-rolling elements (8) in association with the second torque limiting element (7).

However, the torque limiter (19) with built-in coupling system (16), has further advantages in that it allows standard drills (13) or driven members (12) to be used in connection with said torque limiter, and does not require adaptation of existing driven members (12) or drills (13).

In a particular embodiment, the present invention provides a torque limiter (11 or 19) as defined herein above, wherein said second torque limiter element (7) comprises one or more grooves (7a) adapted to receive a first part (8a) of said one or more spring-rolling elements (8) (see FIG. 8A). Alternatively, said grooves may be replaced by any suitable means for accommodating the spring-rolling elements, such as for example, detents, recesses, trenches, . . .

In a further embodiment, the present invention provides the combination of a torque limiter (11) according to this invention, with a sleeve member (1) comprising a hollow portion (2) to embrace a driven member (12) or drill (13). Said torque limiter is adapted to releasably engage said second torque limiter element (7) to said driven member (12) or drill (13) by means of one or more spring-rolling elements (8) in the torque limiter, wherein the built-in mechanism for torque limitation (5) is present on said sleeve member (1) (see FIG. 7A). Said second torque limiter element (7) may be laterally displaced over the first torque limiting element (6) by means of a spring element (6a) wrapped around said first torque limiting element (6).

In said particular embodiment, said sleeve member preferably comprises one or more detents (5b) adapted to receive a second part of said spring-rolling elements (8b). More in particular, said sleeve is preferably a sleeve member (1) according to this invention. Hence, in this embodiment, the hollow sleeve (1) is attached to the drill (13) by means of this particular type of torque limiter (11) as illustrated in FIG. 7A. Hence, within this embodiment the attachment means (4) of the sleeve member are integrated with and consist of the built-in mechanism for torque limitation (5).

In yet a further embodiment, said one or more spring-rolling elements (8) each comprise 2 transmission balls (9) connected by an elastic element (10) such as a coil spring of spring washers, as illustrated in FIG. 8A. Preferably, spring washers (10a) are used, since the elasticity thereof changes very little after initial exposure to high temperatures required for autoclaving.

The force of the elastic elements (10) against the second torque limiting element (7) on the one hand and the driven member (12)/drill (13) (directly) or sleeve member (1) (indirectly) on the other hand, maintains the transmission balls (9) in the groove (7a) of said second torque limiting element (7); or in the detents of said driven member (12)/drill (13) or sleeve member (1) respectively. As the screw or bolt tightens in the object, it exerts a counter-torque on the sleeve member or driven member, which will be transmitted to the transmission balls (9). Once the counter-torque on the transmission balls exceeds the maximum pressure from the elastic element, the counter-torque will cause the transmission balls (9) to disengage from the detents or groove, thus preventing over-tightening of the screw or bolt.

The maximum amount of torque that may be exerted on the screw or bolt is a function of the force exerted by the elastic elements (10) against the transmission balls (9) and is defined by the depth, size and/or shape of the detents in the shaft of the driven member (12)/drill (13) and/or the detents (5b) in the shaft of the sleeve member (1). Thus the maximum amount of torque exerted on the screw or bolt can be adjusted by varying the depth, size and/or shape of the detents in the shaft of the driven member (12)/drill (13) and/or the detents (5b) in the shaft of the sleeve member (1). Hence a single type of torque limiter can be used in association with a set of different driven members (12) or sleeve members (1) each having adjusted detents dependent on the type or size of screw/bolt to be installed. Alternatively the depth, size and/or shape of the groove (7a) in the second torque limiting element (7) may be altered to adjust the maximum amount of torque to be exerted on the screw or bolt, however, in said instance, a separate torque limiter or a separate second torque limiting element according to the invention needs to be used in association with different types of screws or bolts.

In a further embodiment, the driven member (12) may be a drill (13) or a screwdriver, preferably a drill, more in particular a drill which may be embraced by a hollow sleeve, which serves the purpose of a screwdriving element such as a sleeve member (1) according to the present invention.

In yet a further embodiment, all components of the torque limiter (11) according to this invention can be sterilized by autoclaving without affecting the operation of the device. Hence, all of the components of the torque limiter (11 or 19) according to the invention are preferably constructed of stainless steel or other material that will reduce wear and abrasion, can be sterilized and can endure repeated autoclaving.

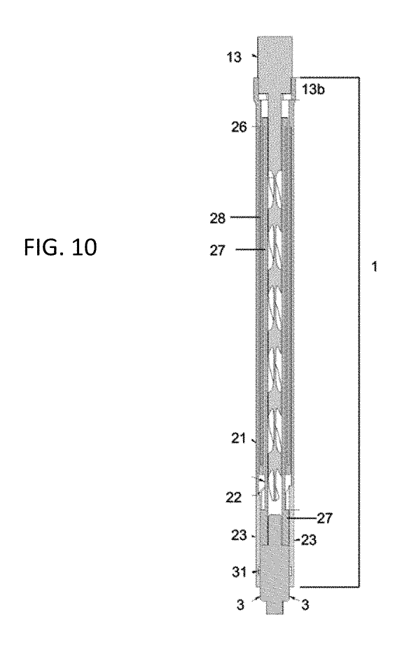

Alternatively, in a third embodiment (20) of the invention (illustrated in FIG. 10), the sleeve member (1) of the present invention may include a built-in mechanism for torque limitation (5) which is based on elastic deformation using a torsional spring. In such embodiment, the sleeve member (1) of the present invention comprises the following elements:
A hollow portion (2) adapted to embrace a drill (3); said hollow portion comprising:
   An external tube (21) having a cam (22)
   An axially moveable sleeve (23) comprising lock pins (24) and release pins (25);
   An elastically deforming body (26) comprising a spline shaft (27) at a first end thereof, which fits within said axially moveable sleeve (23); and a first hollow tube (28) within a smaller second tube (29) at the second end thereof, which fits within said external tube (21); said body (26) is provided with weaknesses (30) which allow adjusting the torsion stiffness of the body (26);
A bolt-or-screw-fitting unit (3) on a first end (1a) of said sleeve member (1); said unit comprising lock connections (31) which may engage with the lock (24) or release pins (25) of the axially moveable sleeve (23)
Attachment means (4) on a second end (1b) of said sleeve member (1) for releasably engaging said sleeve member (1) to a drill (13)

The built-in mechanism for torque limitation (5) in this third embodiment works as flows:

When driving a screw or bolt, the spline shaft (27) will slip with respect to the drive side. The external tube (21) having a cam (22) will not slip with respect to the drive side, at least in as far as the maximum amount of torque is not reached. Hence, the bolt-or-screw-fitting unit (3) will engage with the axially moveable sleeve (23) by means of the lock pins (24) and will drive the screw or bolt. Once the maximum amount of torque is reached, the elastically deforming body (26) will be angularly displaced, thereby allowing the cam (22) to push the axially moveable sleeve (23) forward. As such, the bolt-or-screw-fitting unit (3) will align with the release pins (25) of the axially moveable sleeve (23) thereby no longer being able to drive the screw or bolt.

What is claimed is:

1. A sleeve member comprising:
a hollow portion adapted to embrace a drill,
a bolt- or screw-fitting unit on a first end of the sleeve member, and
attachment means on a second end of the sleeve member, for releasably engaging the sleeve member to the drill or to a motor unit;
wherein the sleeve member comprises a built-in mechanism for torque limitation.

2. The sleeve member according to claim 1, wherein the built-in mechanism for torque limitation is adapted, depending on the type of bolt- or screw-fitting unit in the sleeve member.

3. The sleeve member according to claim 1, wherein the built-in mechanism for torque limitation is defined by one or more breakpoints in the sleeve member.

4. The sleeve member according to claim 3, wherein the resistance of the one or more breakpoints is adapted, depending on the type of bolt- or screw-fitting unit in the sleeve member.

5. The sleeve member according to claim 1, wherein the attachment means are fully integrated in, or correspond with, the built-in mechanism for torque limitation.

6. The sleeve member according to claim 5, wherein the built-in mechanism for torque limitation is defined by one or more detents in the sleeve member, the one or more detents being adapted to receive one or more spring-rolling elements from a torque limiter mounted on the drill or motor unit.

7. The sleeve member according to claim 6, wherein the size and/or form of the one or more detents is adapted, depending on the type of bolt- or screw-fitting unit in the sleeve member.

8. The sleeve member according to claim 6, wherein the one or more detents are asymmetric thereby allowing to apply a different torque limitation in clockwise or counter-clockwise direction.

9. The sleeve member according to claim 1, wherein the sleeve member is made from a plastic material chosen from polyethylene (PE), polypropylene (PP), polystyrene (PS), or polyether ether ketone (PEEK).

10. The sleeve member according to claim 1, wherein the sleeve member is made from stainless steel.

11. The sleeve member according to claim 1, wherein the sleeve member comprises a specific colour-and/or bar-code, each optionally provided on a removable label, to allow for easy identification and/or traceability.

12. A kit comprising the sleeve member according to claim 1, and one or more bolts or screws adapted to fit the bolt- or screw-fitting unit.

13. A torque limiter comprising:
a first torque limiter element capable of embracing at least a first part of a driven member, or a drill;
a second torque limiter element for releasably engaging the first torque limiter element with the driven member or the drill; and
one or more spring-rolling elements for releasably engaging the driven member or drill with the second torque limiter element by means of a built-in mechanism for torque limitation present on the driven member or drill or on the sleeve member according to claim 1, capable of embracing at least a second part of the driven member or drill.

14. A torque limiter comprising:
a first torque limiter element capable of embracing at least a first part of a driven member or a drill;
a second torque limiter element for releasably engaging the first torque limiter element with the driven member or the drill;
one or more spring-rolling elements for releasably engaging the driven member or drill with the second torque limiter element by means of a built-in mechanism for torque limitation present on the driven member or the drill, or on the sleeve member according to claim 1, capable of embracing at least a second part of the driven member or drill;
a coupling system including a coupling bit for coupling the torque limiter to a motor unit; and
a locking system for releasably engaging the driven member or the drill to the torque limiter, by means of a release system.

15. The torque limiter according to claim 13, wherein the second torque limiter element comprises a groove adapted to receive a first part of the one or more spring-rolling elements.

16. The torque limiter according to claim 13, wherein the built-in mechanism for torque limitation comprises one or more detents adapted to receive a second part of the spring-rolling elements.

17. The torque limiter according to claim 13, wherein each of the spring-rolling elements comprises 2 transmission balls connected by an elastic element chosen from one or more spring washers or a coil spring.

18. The torque limiter according to claim 13, wherein the driven member is the drill or a screwdriver.

19. The torque limiter according to claim 13, wherein all components of the torque limiter can be sterilized by autoclaving without affecting the operation of the device.

20. The torque limiter according to claim 13, in combination with a sleeve member comprising:
a hollow portion adapted to embrace a drill,
a bolt- or screw-fitting unit on a first end of the sleeve member, and
attachment means on a second end of the sleeve member, for releasably engaging the sleeve member to the drill or to a motor unit;
wherein the sleeve member comprises a built-in mechanism for torque limitation.

21. The sleeve member according to claim 1 comprising:
a hollow portion adapted to embrace a drill; the hollow portion comprising:
an external tube having a cam,
an axially moveable sleeve comprising lock pins and release pins;
an elastically deforming body comprising a spline shaft at a first end thereof, which fits within the axially moveable sleeve; and a first hollow tube within a smaller second tube at the second end thereof, which fits within the external tube; the body is provided with weaknesses which allow adjusting the torsion stiffness of the body;
a bolt-or-screw-fitting unit on a first end of the sleeve member; the unit comprising lock connections which may engage with the lock or release pins of the axially moveable sleeve;
attachment means on a second end of the sleeve member for releasably engaging the sleeve member to a drill;
wherein the sleeve member comprises a built-in mechanism for torque limitation.
